(19) United States
(12) Patent Application Publication
(10) Pub. No.: US 2022/0008258 A1
(43) Pub. Date: Jan. 13, 2022

(54) HEMOSTATIC MATERIAL AND DEVICE FOR ACHIEVING DURABLE HEMOSTASIS OF A BLEEDING BIOPSY TRACT

(71) Applicant: John T. Watabe, Honolulu, HI (US)

(72) Inventor: John T. Watabe, Honolulu, HI (US)

(21) Appl. No.: 17/363,921

(22) Filed: Jun. 30, 2021

Related U.S. Application Data
(60) Provisional application No. 63/050,741, filed on Jul. 10, 2020.

(51) Int. Cl. A61F 13/36; A61F 13/20; A61F 13/26; A61B 10/00; A61B 10/02

(52) U.S. Cl. CPC A61F 13/36; A61F 13/2031; A61F 13/266; A61B 10/0233; A61F 13/2074; A61B 10/0041; A61F 13/2054

(57) ABSTRACT

Systems and method of facilitating hemostasis in a tract in a patient are disclosed. The method can include, in some cases, delivering an elongate member into the tract, the elongate member comprising a lumen; injecting a suspension through the elongate member, the suspension comprising pledgets comprising surface irregularities; a hemostatic agent; and particles comprising an average diameter less than an average diameter of the pledgets. The pledgets, hemostatic agent, and particles pack the tract and promote hemostasis.

### CROSS-REFERENCE TO RELATED APPLICATIONS

This application claims priority benefit to U.S. Provisional Patent Application No. 63/050,741, filed Jul. 10, 2020, which is hereby incorporated by reference in its entirety.

### BACKGROUND

### Field of the Invention

The invention relates, in some aspects, to achieving hemostasis in a biopsy tract.

### Description of the Related Art

Devices and methods are known for performing a percutaneous needle biopsy of a solid organ. A needle is advanced, typically under image guidance directly in the surface of the solid organ. Current coaxial systems include an inner biopsy needle which passes through an outer needle cannula into the tissue to obtain a tissue specimen. The outer cannula remains positioned in the surface of the organ and allows the operator to take multiple additional biopsy specimens by re-advancing the inner biopsy needle through the cannula to the surface of the organ.

Many biopsy needle designs are available, but most commonly there is a sharp, spring loaded needle which is manually pushed forward into the organ. A hollow side or central chamber in the needle collects the cut tissue specimen and the specimen is then captured by a sleeve or sheath that covers captured specimen. The needle can then be removed along with the captured specimen for collection.

The needle tract created by the biopsy needle is a cylindrical channel often 1 to 2.5 cm in depth. It may transect arteries or veins that traverse this portion of the organ. Arterial bleeding is most likely to cause significant bleeding due to its higher pressures and flow rates compared to veins.

Currently, the main method for controlling bleeding after a biopsy is manual pressure applied at the needle entry skin surface. Hematomas and significant blood loss can rapidly develop when bleeding cannot be controlled by manual compression, especially with kidney biopsies. Post biopsy bleeding can result in prolonged hospitalizations, transfusions, loss of kidney, kidney failure and death. Hematomas and significant blood loss can rapidly develop when bleeding cannot be controlled for other types of biopsies including breast biopsies.

To date there is no universal solution for immediate post renal biopsy bleeding except manual pressure. Achieving hemostasis with manual pressure alone can be challenging since the kidney is often deep to the skin, the kidney moves with each respiration and the biopsy site can be partially under the rib cage. These inherent anatomic challenges contribute to many post biopsy hemorrhages, hospitalizations and even deaths.

Furthermore, arterial pressure in a bleeding biopsy tract can be high, resulting in the expulsion of improperly sized gelfoam pledgets and continued bleeding. This can be immediate or delayed.

Poorly functioning platelets in renal failure patients or lupus anticoagulant can further hamper hemostasis and exacerbate bleeding in many patients.

Developing an effective solution for sealing a kidney biopsy tract has been elusive. Developing an effective solution for sealing a biopsy tract for other types of biopsies has also been elusive.

While many topical hemostatic agents are available for direct application to wounds or surgical incisions, no universally accepted solution has been developed to address post kidney biopsy bleeding or bleeding related to other biopsies when it is refractory to manual pressure.

In other words, needles, cannulas, catheters, and other medical devices can be positioned within tissue for a variety of diagnostic and/or therapeutic indications. However, upon withdrawal of the medical device, bleeding can occur in the tract created by insertion of the device(s), especially in highly vascularized tissues. Conventional techniques such as application of pressure to achieve hemostasis can be ineffective especially in deep, narrow biopsy tracts. Inadequate hemostasis can result in anemia, hematoma formation, and potentially even mass effects such as, for example, compartment syndrome caused by blood pooling in fixed-volume spaces within the body. Improved systems and methods for achieving rapid and durable hemostasis are needed.

### SUMMARY

In some embodiments, disclosed herein are improved hemostatic materials to plug a biopsy tract, including custom sized and/or shaped ridged gelfoam pledgets and a method for applying direct manual pressure directly to the biopsy tract. The particles can be optimized for needle injectability and needle tract conformity, while the addition of one or more clot-promoting agents, e.g., thrombin simultaneously activates the clotting cascade. The ridged or ribbed surface of the plugs can be configured to increase contact points with the biopsy tract wall, increase compaction and reduce extrusion of the plug due to pulsatile arterial pressure. Smaller gelfoam particles added to the mixture serve as packing to fill any dead space.

In some embodiments, a compression device comprises an elongate member, such as, for example, a stiff rod fitted with a concave stopper on its distal end which can be configured to be introduced through the needle lumen to push any residual gelfoam/thrombin onto the biopsy tract surface and apply manual pressure directly to the biopsy tract surface further promoting hemostasis and compacting gelfoam in the tract.

Systems and methods can be configured for a variety of indications, including but not limited to tissue biopsies, including organ biopsies, such as, for example, of the kidney, liver, pancreas, gallbladder, spleen, stomach, lung, heart, brain, breast, uterus, testes, lymph node, bone, blood vessels, and other tissue. In some embodiments, systems and methods can be injectable into a blood vessel, aneurysm, AVM, organ, tumor, or other anatomical site of interest for embolization. For instance, many different types of biopsy may benefit from the embodiments described herein. Each biopsy site may have unique challenges. For breast biopsies, hemotoma can result from a breast biopsy, as well as the risk of infection, skin discoloration, blood loss, and scarring. Managing blood loss can improve the cosmetic outcome of biopsies, as well as prevent unwanted effects of bruising and swelling, thereby increasing patient satisfaction.

In some embodiments, disclosed herein are improved hemostatic materials supported by a compression device to achieve and maintain hemostasis of a bleeding biopsy tract.

In some embodiments, modified cylindrical gelfoam plugs optimize gelfoam packing and retention in the tract.

In some embodiments, raised, ribbed or corrugated surface of the gelfoam plugs provide increased friction points for pledgets to adhere to the wall and promote stability of the plugs within the biopsy tract. The ribbed plugs can be readily stacked on one another and compressed within the tract.

In some embodiments, tight packing can be advantageous to avoid expulsion of the plugs by pulsatile blood and arterial pressure within the tract. Swelling occurs on contact with blood, expanding the gelfoam in the tract further fixing it in place.

In some embodiments, a hemostatic mixture can include additional smaller gelfoam particles or powder configured to fill any dead space not filled by the pledgets and create a tight pack of gelfoam.

In some embodiments, liquid thrombin is added to the mixture of gelfoam plugs and smaller gelfoam particles to promote rapid activation of the clotting cascade and rapid thrombosis, fixing the pledgets in place. In some embodiments, clinical hemostasis can be achieved, for example, in less than about 5, 4, 3, 2, or 1 minute, or less than about 60, 45, 30, 20, 15, 10 seconds, or even less.

In some embodiments, an optimized semi-solid mixture or slurry of gelfoam plugs, smaller particles and liquid thrombin allows unrestricted injectability through a needle, yet enough viscosity to promote rapid hemostasis of an actively bleeding tract.

In some embodiments, a tamp such as, for example a tubular tamp compression rod or plunger can be advanced through the biopsy needle cannula after a biopsy to apply direct manual pressure to the biopsy tract surface. The modified tip of a compression rod can be advanced through the needle cannula to apply direct manual pressure to the needle tract, promoting compaction of gelfoam in the tract and reducing extrusion of the plug from the tract due to arterial pressure. The shaft of the tamp/rod may be marked with measurement indicia, such as, for example, 1 cm marker bands to monitor depth as it is introduced.

In some embodiments, the gelfoam plugs may be modified in a variety of shapes and sizes to improve compaction or wall apposition. This includes, but is not limited to circular, torpedo, cigar, star or twisted shapes.

In some embodiments, the compression rod may be modified to have a hemostatic agent attached or otherwise coupled to its end, as well as another structure, such as an expandable member, e.g., expandable balloon, or an electrocautery device for example. However, some embodiments do not include an expandable member, e.g., expandable balloon or a electromagnetic or other cautery device.

In some embodiments, systems and methods as disclosed herein can advantageously include any number of the following advantages. One advantage can include optimizing goals of rapid occlusion, packing and/or preventing extrusion. Pledgets with increased surface irregularities, including corrugated designs can increase surface irregularity, improve wall apposition/friction, thereby increasing packing and reducing the chance of extrusion. In some cases, a worm like shape and segmented, corrugated design are configured such that each pledget can contract along its long axis within a biopsy tract, promoting increased packing, much like a spring or “slinky” can compress.

The addition of small additional particles can advantageously further increase mechanical packing of the tract, by filling in dead space.

The addition of thrombin can promotes rapid thrombosis of blood in the tract by activating the clotting cascade. Clot solidifies the blood around a scaffold of gel foam plugs and small particles also fixing the entire complex in place.

A series of ring-like crevices, forming circumferential depressions in the surface of each pledget, can result in a corrugated surface of each pledget. Pledgets can have greater surface irregularity to promote improved friction and retention within a biopsy tract.

In some embodiments, disclosed herein is a method of facilitating hemostasis in a tract in a patient, comprising any number of: delivering an elongate member into the tract, the elongate member comprising a lumen; injecting a suspension through the elongate member, the suspension comprising pledgets comprising surface irregularities; a hemostatic agent; and particles comprising an average diameter less than an average diameter of the pledgets, whereby the pledgets, hemostatic agent, and particles pack the tract and promote hemostasis.

In some embodiments, the method further comprises tamping tissue at the distal end of the tract.

In some embodiments, the suspension further comprises saline or water.

In some embodiments, the hemostatic agent comprises thrombin.

In some embodiments, the pledgets comprise gelfoam.

In some embodiments, the particles comprise gelfoam, and/or wherein the particles comprise surface irregularities.

In some embodiments, the pledgets are compressible.

In some embodiments, the pledgets comprises a corrugated surface.

In some embodiments, a pledget further comprises at least 2 corrugations substantially transversely with respect to a longitudinal axis of the pledget.

In some embodiments, the particles are of sufficient number to fill at least about 90% of the volume of the corrugations of the pledgets.

In some embodiments, the method further comprises compressing the pledgets by at least about 25% of their uncompressed volume.

In some embodiments, the volume of the suspension is between about 1 cc and about 5 cc.

In some embodiments, the tract is a biopsy tract, and the method further comprising applying direct mechanical pressure to a tissue surface proximate the distal end of the biopsy tract.

In some embodiments, the tract is a blood vessel.

In some embodiments, the biopsy tract is a kidney biopsy tract. In some embodiments, the biopsy tract is a breast biopsy tract.

In some embodiments, the method also further comprises performing a tissue biopsy procedure.

In some embodiments, disclosed herein is a composition for facilitating hemostasis in a patient, comprising: a plurality of pledgets comprising surface irregularities; a hemostatic agent; and/or particles comprising an average diameter less than an average diameter of the pledgets.

In some embodiments, the hemostatic agent comprises thrombin.

In some embodiments, the pledgets and/or the particles comprise gelfoam.

In some embodiments, the pledgets are compressible.

In some embodiments, the pledgets comprises a corrugated surface.

In some embodiments, the pledgets further comprise at least 2 corrugations substantially transversely with respect to a longitudinal axis of the pledget.

In some embodiments, the particles are of sufficient number to fill at least about 90% of the volume of the corrugations of the pledgets.

In some embodiments, the pledgets are configured to be compressed by at least about 25% of their uncompressed volume.

In some embodiments, the composition is a suspension comprising a volume of between about 1 cc and about 5 cc.

In some embodiments, disclosed herein is a kit for facilitating hemostasis in a patient, comprising: a suspension comprising: a plurality of pledgets comprising surface irregularities; a hemostatic agent; and particles comprising an average diameter less than an average diameter of the pledgets; a tamp; and/or a hollow needle.

In some embodiments, disclosed herein is a suspension for facilitating hemostasis in a patient, comprising: a plurality of gelfoam pledgets comprising surface irregularities comprising between about 3 and about 10 corrugations; a hemostatic agent comprising thrombin; and particles comprising an average diameter less than an average diameter of the pledgets, wherein the particles comprise gelatin and are of sufficient volume to fill at least about 80%, 85%, 90%, 95%, 99%, or more of the space between corrugations.

In some embodiments, a system, kit, and/or method can comprise, consist essentially of, or consist of any number of features as disclosed herein.

### BRIEF DESCRIPTION OF THE DRAWINGS

FIG. 1 is a perspective view of a gelfoam plug with a ribbed or ridged surface, according to some embodiments of the invention.

FIG. 2 is a perspective view of the tubular compression rod with a concave distal tip and pushing platform at its proximal tip according to some embodiments of the invention.

FIG. 3 is a perspective view of dry ridged gelfoam plugs and small gelfoam particles mixed in a syringe according to some embodiments of the invention.

FIG. 4 is a perspective view of a syringe containing liquid thrombin, a syringe containing a mixture of dry gelfoam plugs and smaller gelfoam particles according to some embodiments of the invention, and a 3 way stopcock.

FIG. 5 is a perspective view of a needle cannula embedded in tissue with its tip in a solid organ. FIG. 5 also shows the recently created needle biopsy tract and a syringe containing the Gelfoam plugs, smaller particles and thrombin mixed together, according to some embodiments of the invention.

FIG. 6 is a perspective view of a needle cannula with a compression rod being advanced into the cannula. A concave distal tip on the compression rod is demonstrated. A biopsy tract is depicted with injected gelfoam in the tract, according to some embodiments of the invention.

FIG. 7 is a perspective view of a needle tract filled with gelfoam plugs with smaller gelfoam particles filling in the intervening spaces between the plugs. A tamp rod has been advanced through a needle cannula. A concave tip of the tamp is shown compressing the gelfoam in the biopsy tract and applying manual pressure to ensure hemostasis, according to some embodiments of the invention.

FIG. 8 is a perspective view of the fully inserted compression rod, passing through the needle cannula and applying direct manual pressure to the surface of the biopsy tract according to some embodiments of the invention. A flat, pushing platform is shown on the proximal end of the compression rod.

FIG. 9 illustrates a method of creating the gelfoam plugs using metal casts, according to some embodiments.

FIGS. 10A-10E illustrate steps in a method of promoting hemostasis within a biopsy tract by injecting a hemostatic formulation including particles and/or plugs into the tract.

### DETAILED DESCRIPTION

In some embodiments, disclosed herein is a method for delivering a spongy material to achieve hemostasis in a bleeding biopsy tract after a percutaneous biopsy. The device may also be used for other puncture wounds, tracts or lacerations throughout the body.

In some embodiments, a device can include a tubular foam sponge with a ribbed or ridged surface, but may be any roughened or irregular surface to provide increased contact points or friction with the biopsy tract wall. The foam sponge may be open cell or closed cell design. The sponge material may be any sponge capable of being compressed in a biopsy tract and expanding upon hydration with liquid.

Some non-limiting examples of sponge material include biocompatible gelfoam, but may be non-biocompatible. The surface of the sponge may be modified by adding thrombogenic material or impregnating the sponge with thrombogenic material, including, but not limited to thrombin.

The length of the plugs could be, in some embodiments, about 10 mm with a diameter range from 18 G to 14 G (1.02 to 1.63 mm), in accordance with the most common biopsy needle diameters. But other smaller and larger diameters and lengths could also be used, including those disclosed elsewhere herein.

In some embodiments, a plug could include corrugations, including 2, 3, 4, 5, 6, 7, 8, 9, 10, or more (or ranges including any two of the foregoing values) concentric slit-like crevices that can extend longitudinally, transversely, and/or obliquely with respect to the longitudinal axis of the plug and forming a series of annular depressions in the surface and creating a corrugated, alternating raised and depressed surface (much like the surface of a centipede). While these crevices could be arranged in parallel, they could also be arranged in a myriad of different patterns and shapes including, but not limited to criss-crossed, diagonal, oblique, wavy, angular or curvilinear. The depth, and diameter of the slits could vary and distance between these slits could be constant or vary.

In some embodiments, a plug can include various surface characteristics that could vary, including lobulated, bumpy, spiked, mound-like, or even have a bristled, hairy or fuzzy surface to create friction and adhesion. Any lumpy, bumpy or irregular surface could be used to help embed the plug.

In some embodiments, location and distribution of crevices and raised surfaces: could be uniform (e.g., uniformly regular or irregular) or the lumpy or raised portions of the plug could be located only along the body or end of each plug.

Additional elements: The gelfoam plug could also be supported or enhanced with the addition of other metallic or synthetic supportive elements to improve its wall apposition or structural integrity. Adding an internal helical coil, external struts or protruding spikes could also be employed to enhance packing, provide an internal scaffold, or improved biopsy tract retention and wall apposition.

Other substances: The plugs can be made of a variety of substances and foams. Gelfoam (gelatin foam) is preferred for its proven safety profile, biocompatibility, compressibility, absorption and expansion capabilities. Other substances such as cellulose, gelatin, collagen, fibrin, polyvinyl alcohol, chitosan, polyethylene glycol, glutaraldehyde and other synthetic materials, adhesives and foams, or combinations thereof could also be used.

The modified sponge may also be injected into a vessel through a catheter placed in a blood vessel for purposes of occlusion, or embolization, to stop bleeding from a vessel.

The sponge may be, for example, between about 5 mm and about 10 mm in length by about 1.0 mm in diameter for 18 G needles, 1.3 mm in diameter for 16 G needles, and 1.6 mm in diameter for 14 G needles, or ranges including any two of the foregoing values.

The following definitions will be used herein.

“Pledget” means a piece of absorbable sponge which can be injected through a needle cannula into a biopsy or wound tract.

“Plug” is used interchangeably with pledget in this invention, but describes any piece of preformed material used to fill a biopsy tract. So while sponges are described here, it may be made of any biologic or synthetic material.

“Sponge” means a foam material, in this case gelfoam, which is compressible and adaptable to a biopsy tract. It may be open cell or closed cell design, but preferably open cell to allow hydration, expansion with hydration and compressibility. The sponge is ideally biocompatible and bioabsorbable.

“Hydrate” means to saturate with liquid including, but not limited to saline, thrombin or blood.

“Injectable” means pushing the contents of a syringe through the channel of a needle cannula.

“Needle tract’ means the tubular channel or hole created in tissue by a needle biopsy puncture.

“Tamp” means a tubular rod composed of a proximal round flat disc for pushing the rod forward and a distal concave end to apply pressure to the biopsy tract surface.

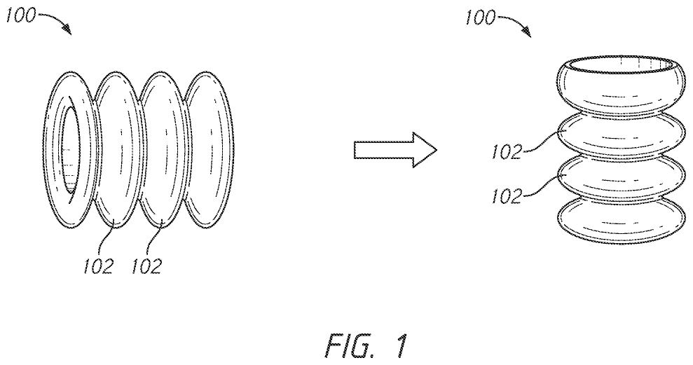

FIG. 1 illustrates a cylindrical gelfoam plug 100 with corrugated or ribbed surfaces 102. The ridges 102 on the pledget 100 can be of variable size, thickness with intervening crevices of variable depth and diameter. The pattern and shapes of the ridges 102 are uniform in the diagram, but may be modified to form irregular or roughened surfaces in various ways to promote increased friction. FIG. 1 is a perspective view of the gelfoam plug 100 with a ribbed or ridged surface.

The ends of the cylindrical plugs 100 are flat and circular in this instance, but could be varied in shape as well, including but not limited to bullet shaped or torpedo shaped.

The diameter of the gelfoam plugs 100 vary from 0.5 mm to about 5 mm, such as between about 1.0 mm to about 2.0 mm, or about 0.5 mm, 1 mm, 1.5 mm, 2 mm, 2.5 mm, 3 mm, 3.5 mm, 4 mm, 4.5 mm, 5 mm, or more or less, or ranges including any two of the foregoing values, depending on the size of the biopsy needle used. Typical non-limiting examples of biopsy needle diameters are 14 G, 16 G, and 18 G.

The length of the gelfoam plugs 100 can vary from, in some cases, between about 0.25 cm to about 2 cm, or between about 0.5 cm to about 1.0 cm.

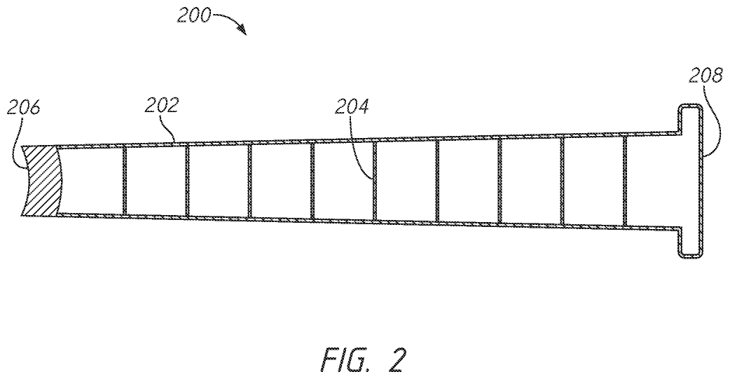

FIG. 2 illustrates a tamp 200, according to some embodiments of the invention. The shaft of the rod 202 can be made of non-breakable, stiff material and can be marked with measurement indicia 204, e.g., 1 cm markers for determining its depth. The shaft 202 may be made of, for example, metal, hard plastic, wood or other synthetic material. The tamp 200 can have a blunt, atraumatic distal end 206 in some cases. FIG. 2 is a perspective view of the tubular compression rod 200 with a concave distal tip 206 and pushing platform 208 at its proximal tip.

The distal tip 206 can be concave in this design, but may also be convex or round. The distal end 206 may be attached to an expandable member, such as a balloon for compression, an electrocautery device, and/or a hemostatic plug. There may be one, two, or more inner channels through the tamp 200 to inject liquid through it.

The proximal end 208 can be a round flat platform for pushing the rod forward with a thumb, palm or digit. It may be other shapes, and it may have a concave or convex surface or irregular surface, so long as it allows for pushability of the tamp 200.

The markers or indicia 204 that can be present on the side of the tamp 200 are designed at one cm intervals, but could be any length intervals. Markers are not essential for this device to achieve hemostasis, but provide a convenient method of monitoring depth of the rod as it is being advanced.

The diameter of the tamp 200 can be identical or substantially identical to the diameter of the inner channel of the biopsy needle.

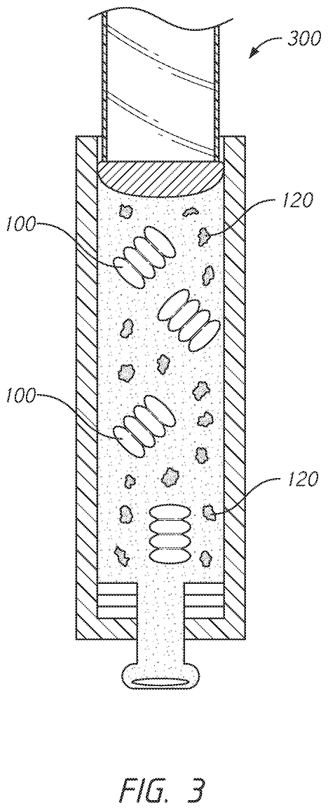

FIG. 3 is a perspective view of dry ridged gelfoam plugs 100 and small gelfoam particles 120 mixed in a syringe 300 according to some embodiments of the invention. FIG. 3 is a perspective view of dry ridged gelfoam plugs 100 and small gelfoam particles 120 mixed in the syringe 300.

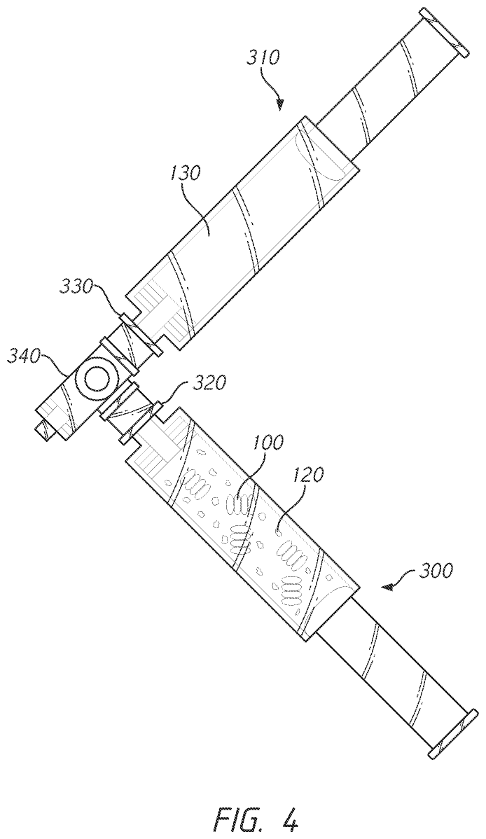

FIG. 4 illustrates one syringe 310 filled with reconstituted liquid thrombin 130. Human recombinant thrombin is preferred. Another syringe 300 is filled with gelfoam plugs 100 and smaller gelfoam particles 120. FIG. 4 is a perspective view of the syringe 310 containing liquid thrombin 130. FIG. 4 is a perspective view of the syringe 300 containing a mixture of dry gelfoam plugs 100 and smaller gelfoam particles 120. FIG. 4 is a perspective view of a 3 way stopcock 310.

In the diagram, the two syringes 300, 310 are attached to different ports 320, 330 on the 3 way stopcock 340. Alternatively, the syringes 300, 310 could be fitted with alternating male-female ends to directly mix the thrombin 130 and gelfoam 100, 120 with each other.

The smaller gelfoam particles 120 can be regular or irregular in shape, measuring up to 1 mm in diameter or more or less. They can be variable in shape and surface contour.

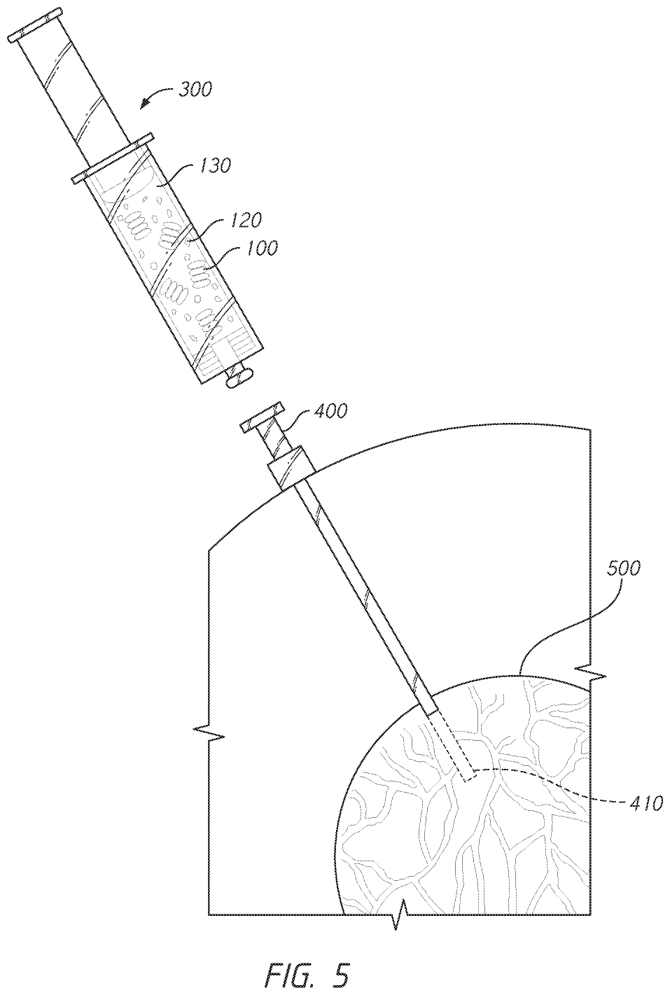

FIG. 5 illustrates a needle cannula 400 in the body after a biopsy with a cylindrical biopsy tract 410 in the biopsied tissue. The gelfoam and thrombin mixture 100, 120, 130 is loaded and mixed in a syringe 300 and about to be attached to the needle cannula 400 by turning the threads on the syringe 300 onto the end of the needle cannula 400. FIG. 5 is a perspective view of the needle cannula 400 embedded in tissue with its tip in a solid organ 500. FIG. 5 represents the recently created needle biopsy tract 410. FIG. 5 shows the syringe containing the Gelfoam plugs 100, smaller particles 120 and thrombin 130 mixed together.

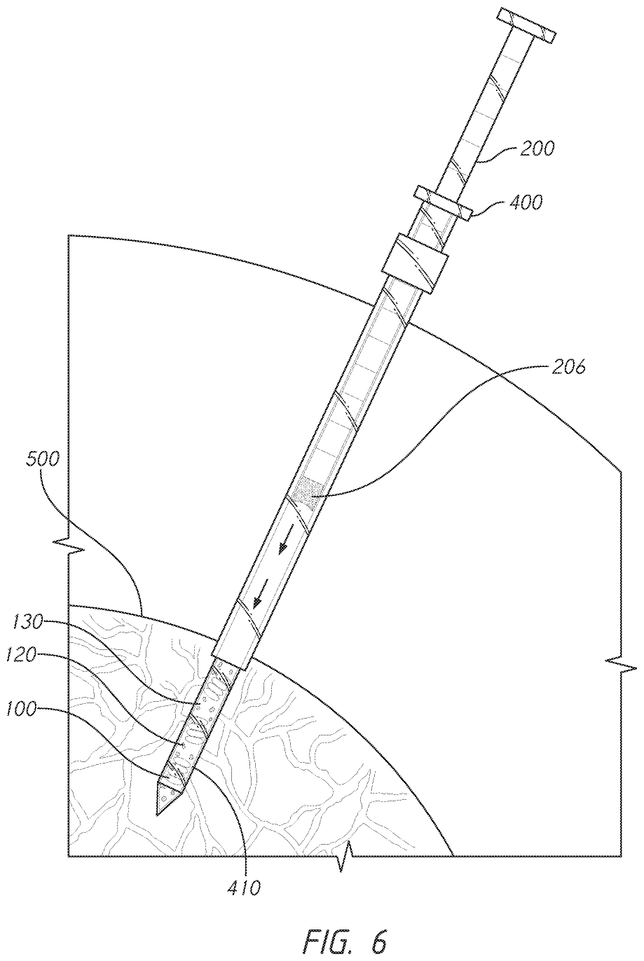

FIG. 6 shows the injected of the mixture of gelfoam plugs 100, small particles 120 and thrombin 130 packed in the biopsy tract (D). FIG. 6 also illustrates the tamp 200 being advanced into the needle cannula. FIG. 6 is a perspective view of a needle cannula 400 with a compression rod 200 being advanced into the cannula 400. A concave distal tip 206 on the compression rod 200 is demonstrated. The biopsy tract 410 is depicted with injected gelfoam 100, 120 in the tract 410.

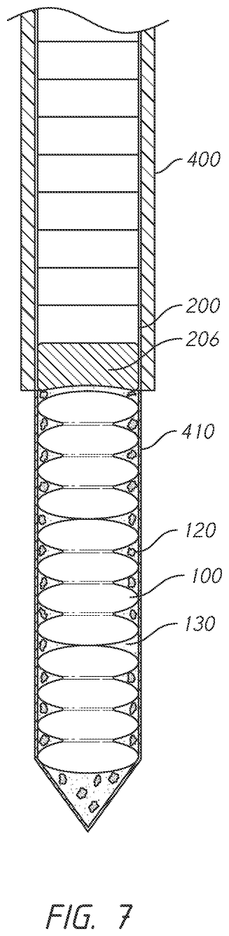

FIG. 7 illustrates the injected gelfoam 100, 120 packed in the needle tract 410. The gelfoam plugs 100 fill the tract lumen and the smaller gelfoam particles 120 fill adjacent dead space between the plugs 100. FIG. 7 is a perspective view of a needle tract 410 filled with gelfoam plugs 100 with smaller gelfoam particles 120 filling in the intervening spaces between the plugs 100. The tamp rod 200 has been advanced through a needle cannula 400. The concave tip 206 of the tamp 200 is shown compressing the gelfoam 100 in the biopsy tract 410 and applying manual pressure to ensure hemostasis.

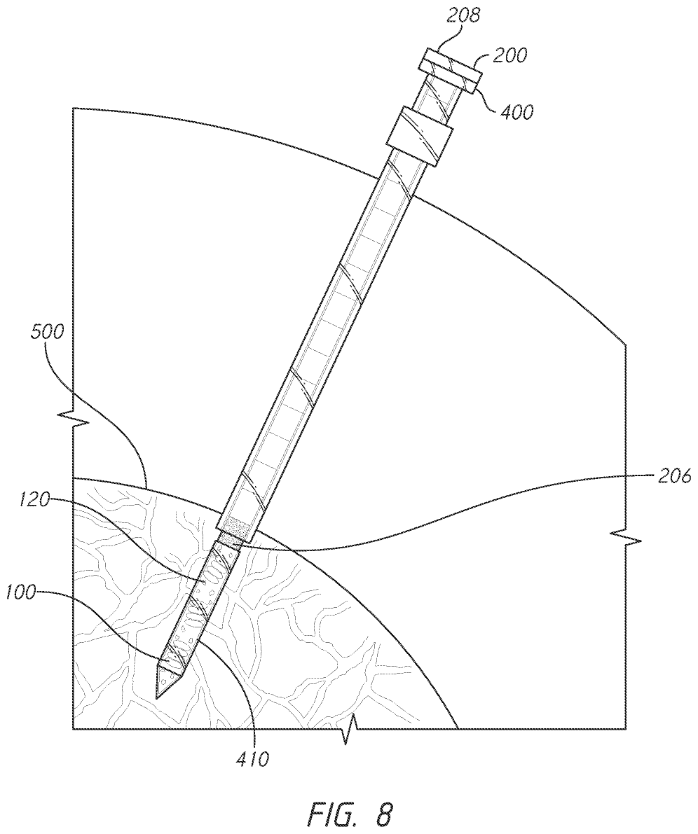

FIG. 8 illustrates a tamp device 200 advanced through the needle cannula 100 and its distal tip 206 applying direct pressure to the biopsy tract surface, thereby compacting the gelfoam 100, 120 in the tract and preventing extrusion of gelfoam 100, 120 by pulsatile blood. FIG. 8 is a perspective view of the fully inserted compression rod 200, passing through the needle cannula 400 and applying direct manual pressure to the surface of the biopsy tract 410. The flat, pushing platform 208 is shown on the proximal end of the compression rod 200.

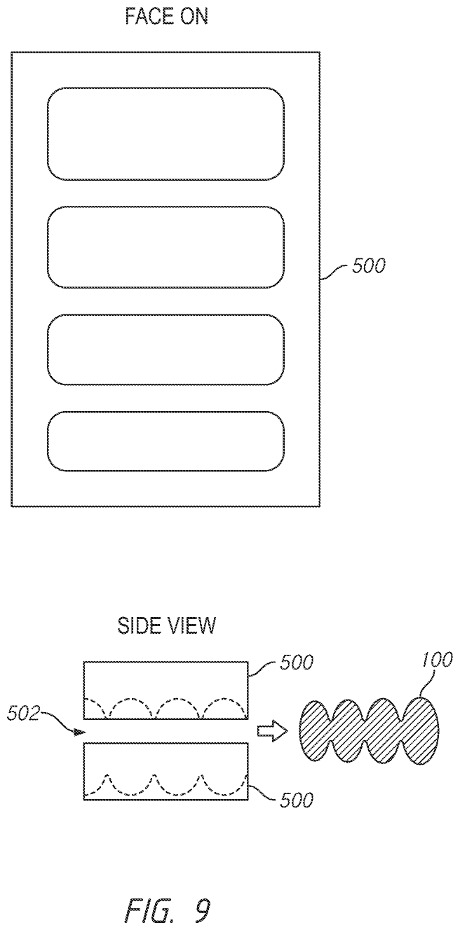

FIG. 9 illustrates a method of creating the gelfoam plugs 100 using metal casts 500. Hollow inner chamber 502 could be created between two mirror image casts 500. When the hollow casts 500 are opposing each other, the shape of the cylindrical ribbed pledgets 100 can be formed by pouring in the gelfoam into the casts to create the desired shape. FIG. 9 illustrates a method of creating the gelfoam plugs 100 using metal casts 500.

Gelfoam is a biocompatible commercially available sponge that expands when hydrated with fluid such as saline or blood. This makes it highly advantageous for injecting into a needle tract. Its occluding properties can be enhanced through shaping or modifying its surface characteristics.

A proper combination of gelfoam sponge 100, 120 and liquid such as thrombin 130 (including thrombin precursors such as prothrombin in some cases) can be advantageous in some cases to optimize filling of a needle biopsy tract and minimize excess liquid. In such an embodiment, the thrombin 130 can be prepared from initial blood composition. The blood composition can be whole blood or blood fractions, e.g., a product of whole blood such as plasma. Thrombin 130 can be autologous, human including pooled plasma, or of non-human source. It is also possible that the thrombin 130 is prepared by recombinant methods.

The total volume of solution can be varied, such as, for example, between about 1 cc and 5 cc for a typical biopsy tract. However, this could be modified for other applications, such as about, at least about, or no more than about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25 cc, or more or less, or ranges including any two of the foregoing values. The final mixture could be variable viscosity and thickness depending on combination of pledgets, particles and volume of liquid.

The thrombin 130 can comprises calcium chloride as an additional active ingredient. The concentration of thrombin 130 in the solution can be, for example, in the range of from about 2 to about 15,000 IU/ml, in the range from about 2 to about 4,000 IU/ml, or in the range of from about 4,000 to about 10,000 IU/ml, or about 1,000 IU/ml although more dilute or concentrated solutions can be used.

Calcium chloride concentration in the solution can be in the range of from about 2 to about 6.2 mg/ml, or in the range of from about 5.6 to about 6.2 mg/ml, such as in the concentration of 5.88 mg/ml. The thrombin 130 may also comprise excipients. As used herein the terms “excipient” refers to an inert substance which is added to the solution. The excipients can be added into the solution in order to ensure that the active ingredient retains its chemical stability and biological activity upon storage, or for aesthetic reasons e.g. color. Examples of excipients include, but are not limited to, human albumin, mannitol and sodium acetate. The human albumin in the solution can be in the range of from about 2 to about 8 mg/ml. Mannitol can be in the concentration range of from about 15 to about 25 mg/ml. Sodium acetate can be added to the solution in the range of from about 2 to about 3 mg/ml. The thrombin can also comprise carriers such as water or saline for injection.

In some embodiments, the plugs 100 could be cylindrical, but shapes include but are not limited to star shaped, corkscrew, helical, cubed, spherical, triangular, polygonal, peanut, popcorn, multi-faceted, multi-pronged or conical shaped. Just as conventional packing materials come in many varied shapes, any shape that could be used to fill up the tract could be used.

The plugs or pledgets 100 could be loaded in a syringe 300 in a dry form prior to being hydrated. The number of plugs 100 and mixture of plugs 100 and smaller particles 120 could vary.

Gelfoam is compressible as it is a foam derived from porcine gelatin. In some embodiments, a pledget 100 can be compressible to about or at least about 90%, 80%, 70%, 60%, 50%, 40%, 30%, 20%, 10%, or more or less of its uncompressed volume, and ranges including any two of the foregoing values. The gelfoam could be compressed or non-compressed.

Additional small particles 120 can be added to the plugs/pledgets 100 to fill in any dead space. For example, 0.25 cm to 3 mm, e.g., 0.5 to 1.0 mm particle sizes could be used, but this could vary with the size of the biopsy tract. Particles 120 could include a variety of shapes, including but not limited to star shaped, cylindrical, cubed, polygonal, or other shapes. Surfaces of the particles could be, for example, irregular, fuzzy, bumpy or smooth. In some embodiments, an average diameter of the particles 120 can be about or less than about 90%, 85%, 80%, 75%, 70%, 65%, 60%, 55%, 50%, or less of the average diameter of the pledgets 100, or ranges including any two of the foregoing values.

The tamp 200 could include a tubular rod could be advanced through the biopsy cannula 400 to provide mechanical pressure or electrocautery to the biopsy tract 410, further promoting lasting hemostasis. Shaft diameters 202 can match that of the biopsy needle, generally between 14 G and 18 G, but can be any diameter. The shaft length 202 can be long enough to protrude beyond the tip of the biopsy cannula. The distal tip 206 and/or another region of the tamp 200 could be fitted with a hemostatic agent, rubber or synthetic tip, electrocautery tip, balloon or other modification to provide manual pressure or induce hemostasis. To apply pressure to the biopsy tract 410 after gelfoam thrombin injection 100, 120, 130, the tip 206 may be flat, concave or blunted. The shaft 202 could be marked 204, e.g., labeled with 1 cm or other markers to monitor depth of the tip 206 as it is advanced through the needle cannula 400.

Proper sizing can also be advantageous. In some embodiments, too large a particle 120 will hamper needle injectability. Too narrow particles 120 may be readily extruded by pulsatile bleeding, resulting in continued bleeding.

Roughened or corrugated surface of the plugs 100 may help maintain their position in the tract.

Smaller particles or powders 120, such as, for example, less than about 2, 1.5, 1, 0.8, 0.7, 0.6, or 0.5 mm (or ranges including any two of the foregoing values) particles or powder added to the mixture of pledgets 100 and thrombin 130 could help pack more gelfoam into the biopsy tract 410 and fill dead space, further contributing to occlusion of the tract 410 in some embodiments.

In some aspects, bleeding biopsy tract can be optimized by using any number of methods of hemostasis including but not limited to: (1) mechanical blockage of the tract; (2) simultaneous activation of the clotting cascade; and (3) direct manual pressure to the biopsy site. Ribbed or ridged pledgets 100 increase the number of contact points and friction with the tract wall, promoting greater wall apposition of the pledgets 100. The addition of a manual pressure device 200 directly to the tract further promotes hemostasis even when tissues are deep to the skin and could prevent extrusion of pledgets from the tract.

Arterial pressure in the biopsy tract 410 can be high with kidney biopsies and the velocity of the blood pulsating out the tract can be brisk, requiring rapid occlusion with properly sized plugs to immediately and firmly seal the tract. Randomly cut gelatin sponge pledgets lack specificity and conformity for the needle tract and require time for the own body's clotting cascade to activate and form clot around the pledgets. Adding ridges or corrugations to the plug's surface 100 increases friction points to promote wall apposition. In addition, adding smaller particles 120 to fill dead space promotes complete and rapid thrombosis of the tract. It can also be beneficial to add a clotting agent such as thrombin 130 to simultaneously clot blood in the tract, creating a firm clot around a Gelfoam scaffold. Applying direct pressure to the biopsy tract 410 also minimizes extrusion of the plug 100 and provides additional hemostasis where it is most needed, at the surface of the bleeding organ rather than at the skin surface. Without these measures, there is a risk that the occluding agent can be pushed out of the tract by pulsating blood before a stable clot can form.

In some embodiments, a kit for hemostasis can include any number of the following components: Gelfoam pledgets 100: e.g., ridged or ribbed pledgets with raised edges around its sides; additional Gelfoam powder/particles 120 that can have a particle diameter less than that of the pledgets 100, such as at least about 10%, 20%, 30%, 40%, 50%, 60%, 70%, 80%, 90%, or more or less of the volume of the pledgets 100, or ranges including any two of the foregoing values. In some embodiments, the total volume of dry Gelfoam plugs and particles 100, 120 in a 3 cc syringe 300 is mixed with 3 cc thrombin solution 130; total volume 3 cc. Another component can include, for example, thrombin 130 (human recombinant) 3000 units, mixed in 3 cc of solution. Another component can include a stopcock 340, such as a 3 way stopcock. Another component can include a balloon tamp/plunger 200, e.g., a cylindrical rod, sized to the diameter of needles, cupped/concave soft plastic stopper 206 (like a syringe stopper, but concave instead of convex). The length can be, for example, 8, 9, 10, 11, 12, 13, 14, 15, or 16 cm long or more or less with 1 cm markers 204 on the shaft 202. In some embodiments, a needle can be 18 G, 16 G, 14 G, or more or less in diameter, with a pusher on back end to apply forward pressure with thumb. The stopper at the tip of the rod may have one or more of a hemostatic plug, electrocautery device or small balloon at its tip. A hemostatic plug can include, for example, hemostatic patches and other topical and injectable hemostatic agents. Hemostatic patches could include, for example, an occlusive path made of CHITO-SEAL, D-STAT, kelp, PEG-covered oxidized cellulose (PCOC), PEG covered collagen (PCC), or combinations thereof. A tamp 200 can include a tubular rod, pusher fitted with a cupped or concave distal end 206 with or without a hemostatic plug or material on it.

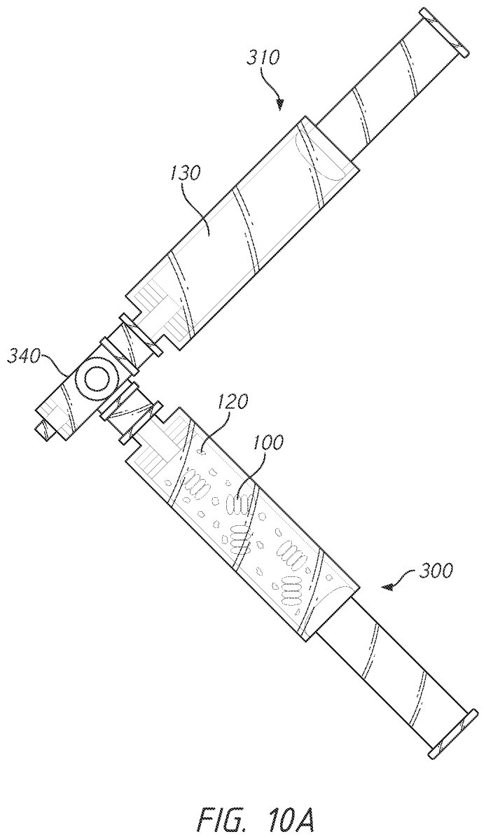

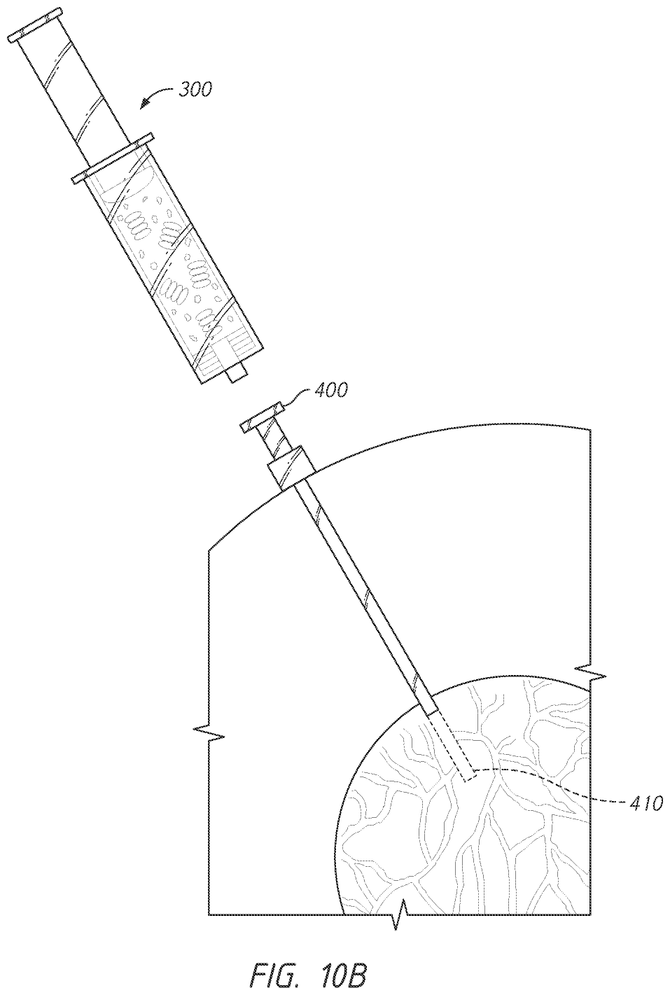

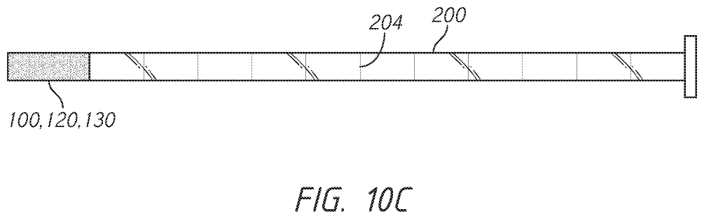

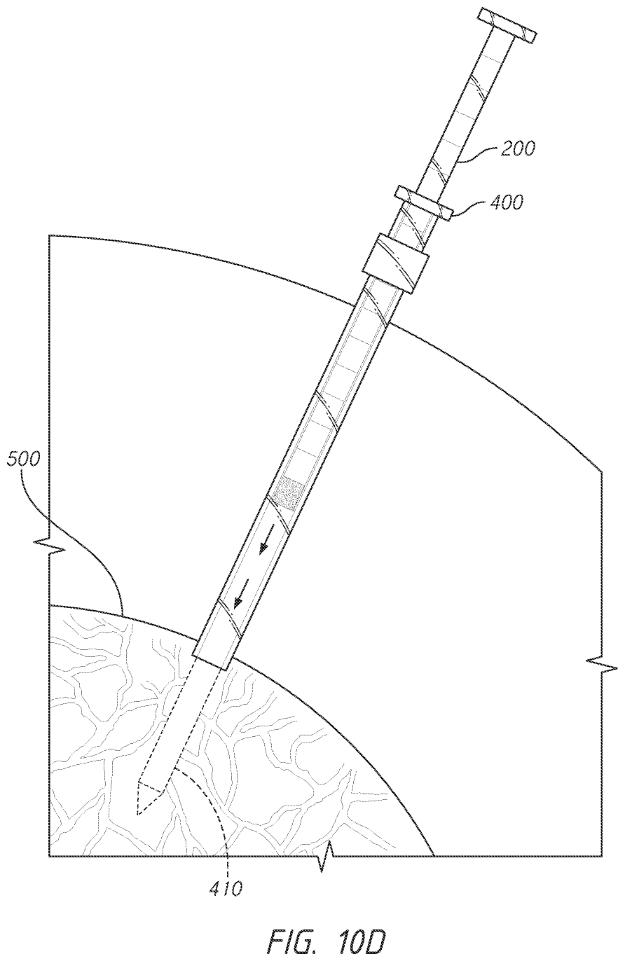

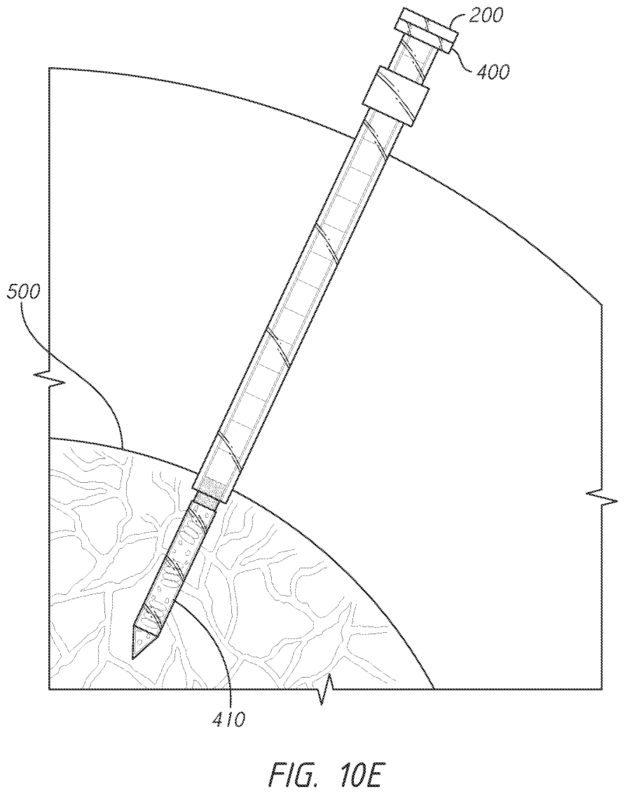

FIGS. 10A-10E illustrate steps in a method of promoting hemostasis within a biopsy tract 410 by injecting a hemostatic formulation including particles 120 and/or plugs 100 into the tract 410. Methods of performing hemostasis are also disclosed. In some embodiments, a method includes any number of the following: reconstitute lyophilized thrombin 130 in container with 3 cc sterile water by aspirating water with a syringe and injecting it into the thrombin vacuum packed container; aspirate liquid thrombin 130 with a 5 cc sterile syringe 310 and needle; attach thrombin containing syringe 310 to a standard 3-way stopcock 340; attach prefilled syringe 300 containing 3 cc pre-cut Gelfoam ridged pledgets 100 and particles 120 to the other port on the 3-way stopcock 340 (FIG. 10A); mix the thrombin 130 and gelfoam 100, 120 by depressing one syringe while aspirating the other syringe until a liquid suspension of Gelfoam pledgets and thrombin are formed 100, 120, 130; perform biopsies using a coaxial biopsy needle. Remove the inner biopsy needle, leaving the outer cannula 400 in place (FIG. 10B); attach thrombin gelfoam suspension syringe 300 to the outer needle cannula 400 and inject 3 cc of the mixture firmly and steadily into the needle tract 410 or until resistance is met. If bleeding persists, inject another dose; if bleeding is no longer seen from the needle hub, immediately insert tamp 200 into the outer needle cannula 400 and depress tamp 200 until it is flush with the end or the cannula or just beyond it (FIG. 10D). Use the 1 cm markers 204 on the tamp 200 to determine depth. Hold manual pressure by applying gentle forward pressure on the cannula 400 and tamp 200 simultaneously for up to 5 minutes to compress the tract 410 and provide additional hemostasis (FIG. 10E); remove tamp 200 and check for bleeding; if bleeding continues, repeat with an additional 2.5 cc injection. FIG. 10C illustrates an embodiment of packed gelfoam soak with thrombin 100, 120, 130, according to some embodiments.

Another method is described herein. Gelfoam plugs 100 and small gelfoam particles 120 are supplied in a dry form in a 5 cc syringe 300. The total volume of gelfoam is approximately 3 cc, but can be smaller or greater ranging from 1-5 cc, or more or less. The 3,000 units lyophilized thrombin 130 is reconstituted in 2 cc sterile water, using sterile technique. The volume of thrombin can be modified between 1 and 5 cc or more or less and total dose can be modified between 1000 units and 5000 units or more or less. The gelfoam 100, 120 is mixed with thrombin solution 130, forming a thick mixture of thrombin soaked pledgets and particles 100, 120, 130.

Once the mixture is made, biopsy samples are taken from the site, such as the kidney or breast for example. Upon bleeding from the needle hub, the syringe 300 containing thrombin and gelfoam 100, 120, 130 are injected is attached to the needle cannula 400, then injected. The syringe 300 is removed to monitor for further bleeding from the tract. If there is further bleeding, the last 1 cc of the mixture could be injected.

The tamp 200 could be advanced through the needle 400 until it protrudes just beyond the end of the cannula to apply direct manual pressure to the tract 410.

FIGS. 10A-E illustrate steps in a method of promoting hemostasis within a biopsy tract by injecting a hemostatic formulation including particles 120 and/or plugs 100 into the tract 410 that can include steps as described, for example above.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “promoting hemostasis in a biopsy tract” includes “instructing the promotion of hemostasis in a biopsy tract.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. A method of facilitating hemostasis in a tract in a patient, comprising:
delivering an elongate member into the tract, the elongate member comprising a lumen;
injecting a suspension through the elongate member, the suspension comprising pledgets comprising surface irregularities; a hemostatic agent; and particles comprising an average diameter less than an average diameter of the pledgets,
whereby the pledgets, hemostatic agent, and particles pack the tract and promote hemostasis.

2. The method of claim 1, further comprising tamping tissue at the distal end of the tract.

3. The method of claim 1, wherein the suspension further comprises saline or water.

4. The method of claim 1, wherein the hemostatic agent comprises thrombin.

5. The method of claim 1, wherein the pledgets comprise gelfoam.

6. The method of claim 1, wherein the particles comprise gelfoam, and/or wherein the particles comprise surface irregularities.

7. The method of claim 1, wherein the pledgets are compressible.

8. The method of claim 1, wherein the pledgets comprises a corrugated surface.

9. The method of any of claim 8, further comprising at least 2 corrugations substantially transversely with respect to a longitudinal axis of the pledget.

10. The method of claim 8, wherein the particles are of sufficient number to fill at least about 90% of the volume of the corrugations of the pledgets.

11. The method of claim 1, further comprising compressing the pledgets by at least about 25% of their uncompressed volume.

12. The method of claim 1, wherein the volume of the suspension is between about 1 cc and about 5 cc.

13. The method of claim 1, wherein the tract is a biopsy tract, and the method further comprising applying direct mechanical pressure to a tissue surface proximate the distal end of the biopsy tract.

14. The method of claim 1, wherein the tract is a blood vessel.

15. The method of claim 1, wherein the biopsy tract is a kidney biopsy tract.

16. The method of claim 1, wherein the biopsy tract is a breast biopsy tract.

17. The method of claim 1, further comprising performing a tissue biopsy procedure.

18. A composition for facilitating hemostasis in a patient, comprising:
a plurality of pledgets comprising surface irregularities;
a hemostatic agent; and
particles comprising an average diameter less than an average diameter of the pledgets.

19-41. (canceled)

42. A suspension for facilitating hemostasis in a patient, comprising:
a plurality of gelfoam pledgets comprising surface irregularities comprising between about 3 and about 10 corrugations;
a hemostatic agent comprising thrombin; and
particles comprising an average diameter less than an average diameter of the pledgets,
wherein the particles comprise gelatin and are of sufficient volume to fill at least about 95% of the space between corrugations.

43. (canceled)

44. (canceled)
